(19) United States
(12) Patent Application Publication
(10) Pub. No.: US 2005/0080205 A1
(43) Pub. Date: Apr. 14, 2005

(54) Method for producing polyoxyalkylene glycols

(75) Inventors: Kai-Uwe Baldenius, Ludwigshafen (DE); Arnulf Lauterbach, Ludwigshafen (DE); Hans-Peter Seelmann-Eggebert, Limburgerhof (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/503,572

(22) PCT Filed: Dec. 19, 2003
(86) PCT No.: PCT/EP03/14591

(30) Foreign Application Priority Data
Dec. 20, 2002 (DE) 102 61 230.7

(51) Int. Cl. C08G 59/14; C07C 41/03

(52) U.S. Cl. 525/524; 568/679

(57) ABSTRACT

The present invention relates to a process for preparing polyoxyalkylene glycol of high purity and having a low color number from the corresponding alkylene glycol and a starter in the presence of a basic catalyst, wherein a reducing agent is present in the polymerization.

The present invention relates to a process for preparing polyoxyalkylene glycols by polymerizing the corresponding alkylene glycols in the presence of a reducing agent. This results in a polyoxyalkylene glycol, especially a polyethylene glycol, of high purity and having low color numbers.

One route to the preparation of polyoxyalkylene glycols is the polymerization of the corresponding alkylene oxides using basic catalysts, for example hydroxides or alkoxides of the alkali metals and alkaline earth metals. An alcohol is also added as a starter, and then addition reaction of the alkylene oxide to the starter takes place.

Examples of polyoxyalkylene glycols are polyethylene glycol PEG, polypropylene glycol PPG and polybutylene glycol PBG, which are prepared from ethylene oxide EO, propylene oxide PO and butylene oxide BO respectively. Also known are mixed polymers of EO, PO and/or BO, for example EO with PO. The mixed polymers may be random polymers or block copolymers.

Polyoxyalkylene glycols have highly varied fields of application. In many, high requirements are placed on the purity and color number of the polyoxyalkylene glycol, for instance in products which are taken by humans, for example in foods and pharmaceutical products. The polyoxyalkylene glycol most frequently used in these fields is PEG.

The requirements on PEG used in pharmaceutical products are defined in highly varied pharmacopoeias, for example the Deutsche Arzneimittelbuch [German Pharmacopoeia] DAB, the US Pharmacopoeia USP and the European Pharmacopoeia EUP. For instance, according to USP, the PEG has to be colorless, and according to EUP, the maximum color number as a 25% solution in water is 20 APHA. Examples of further requirements are a maximum water content of 0.2%, a maximum content of monoethylene glycol and diethylene glycol together of 0.25%, a maximum content of sulfate ash of 0.1% and a maximum aldehyde content (expressed as HCHO) of 30 ppm.

To industrially prepare polyoxyalkylene glycols, in particular PEG, which meet the high requirements in the foods and pharmaceuticals sector, the starting products, including polyoxyalkylene glycols, are generally of high purity. This requires a costly and inconvenient prepurification of the reactants and is therefore costly. There exist only a few patent applications relating to the preparation of polyoxyalkylene glycols from alkylene glycols of technical grade quality.

EP-A 1 245 608 describes the use of triethylene glycol TEG for the preparation of polyethylene glycols to achieve a low content of monoethylene glycol MEG and diethylene glycol DEG. This results in a PEG having low MEG and DEG contents, although no information is given with regards to further requirements. In particular, not even the color number of the products obtained is mentioned.

RO-A 62314 describes the preparation of tetraethylene glycol from TEG and EO under base catalysis. For purification, the product has to be distilled.

JP-A 53 046 907 describes the catalytic hydrogenation of polyalkylene oxides to reduce the color number in the products.

It can be stated that hitherto there exist only a few processes which can be used on the industrial scale and allow polyoxyalkylene glycols of high purity to be prepared from alkylene glycols of technical grade quality. These processes frequently deliver a certain polyoxyalkylene glycol in the desired high purity, but other polyoxyalkylene glycols are not obtainable in the desired purities, if at all.

It is an object of the present invention to provide a process for preparing polyoxyalkylene glycols, in particular PEG, which starts from alkylene glycols of technical grade purity and deliver the desired products in qualities which satisfy the high requirements on color and purity. The process should be usable universally. The polyoxyalkylene glycols or the PEG should preferably satisfy the requirements in the foods and pharmaceutical industry. In particular, the requirements laid down in different pharmacopoeias should be fulfilled.

We have found that this object is achieved by a process for preparing polyoxyalkylene glycol of high purity and having a low color number from the corresponding alkylene oxide and a starter in the presence of a basic catalyst, wherein a reducing agent is present in the polymerization.

The polyoxyalkylene glycol is preferably PEG.

It has been found that the presence of a reducing compound during the polymerization reaction allows polyoxyalkylene glycols of high purity and low color number to be obtained.

Preference is given to adding the reducing agent before the polymerization. However, it can also be added during the polymerization.

It is possible to use the customary reducing agents which are known to those skilled in the art. Examples include complex hydrides, for example borohydrides and aluminohydrides, preferably LiAlH4, NaBH4, LiBH4 and KBH4, BH3, alkylboranes and hydrogen in combination with hydrogenation catalysts known to those skilled in the art and also mixtures of the reducing agents mentioned. Greater preference is given to borohydrides, particular preference to KBH4 or NaBH4 and mixtures thereof.

The reducing agent is used in amounts of from 0.002 to 0.06% by weight, preferably from 0.002 to 0.02% by weight, in particular from 0.004 to 0.02% by weight. It can be used in the form of a solid or as a solution or suspension in a suitable solvent. Suitable solvents are known to those skilled in the art, and examples include the alkali-stabilized solution, tertiary alcohols, secondary alcohols, for example isopropanol, or else primary alcohols such as methanol and ethanol. The alcohol used as a starter can also serve as the solvent.

Preference is given to adding the reducing agent in the form of a solution.

According to the invention, highly differing starters can be used, and their use depends on the polyoxyalkylene glycol to be obtained. Examples of suitable starters include monoethylene glycol MEG, diethylene glycol DEG, triethylene glycol TEG, monopropylene glycol MPG, dipropylene glycol DPG, tripropylene glycol TPG, monobutylene glycol and dibutylene glycol.

Suitable basic catalysts are known to those skilled in the art and are generally selected from hydroxides and alkoxides of the alkali metals and alkaline earth metals. It is added in an amount of from 0.001 to 5% by weight, preferably from 0.01 to 1% by weight. According to the invention, the catalysts are generally used in combination with the reducing agent, and can be added in form of a mixture with the starter or reducing agent or separately to the reaction mixture to be reacted.

As an alternative to the separate use of reducing agent and catalyst, it is possible to use strongly basic reducing agents which generate alkoxides in situ. Examples of such basic reducing agents include LiAlH4, KAlH4 and NaBH4 in alkaline-stabilized aqueous solution, preferably with NaOH or KOH.

The reaction of the starter with alkylene oxide is generally carried out in such a way that starter and catalyst and/or the basic reducing agent are mixed before the addition of alkylene oxide, optionally dewatered and brought to the reaction temperature above 80° C. The alkylene oxide is then added. Once the reaction abates, the mixture is cooled and drained from the reactor. Preference is given to carrying out the reaction in a temperature range between 105 and 180° C., more preferably between 115 and 160° C.

It is suspected that the high color numbers of polyoxyalkylene glycols which result when starters of technical grade quality are polymerized are caused by the presence of aldehydes. As the examples show, the high color numbers of the polyoxyalkylene glycols obtained by polymerization of starters of technical grade quality correlate with the amount of the carbonyl function in the starter. The addition of the reducing agents used according to the invention reduces these carbonyl functions (aldehydes and ketones) and thus achieves low color numbers.

Preference is given to using the present invention to prepare polyethylene glycol PEG of high purity and low color number from starter of technical grade quality. Greater preference is given to the starter used being triethylene glycol TEG. In such a case, a PEG is obtained which has not only a low color number but also a small amount of MEG and DEG and is therefore suitable in principle for use in foods and pharmaceutical products.

In particular, the process according to the invention is used, in order to prepare PEG from ethylene oxide using TEG as the starter, said PEG having a molecular weight of from 150 to 500 mol/g, preferably from 190 to 300 mol/g, in particular from 190 to 250 mol/g. Industrial scale processes which deliver PEG having a molecular weight of ≦500 g/mol and of the quality achieved in accordance with the invention from starters of technical grade quality do not yet exist.

The color number achieved by the process according to the invention depends on the purity of the starting products and also on the amount of reducing agent. When preparing PEG, the process according to the invention allows the use of starters having carbonyl contents of >25 ppm. According to the invention, it is possible to achieve color numbers of <20 APHA, which means that an appropriate PEG achieves the requirements of the European Pharmacopoeia. Adjustment of the reaction conditions and of the starting products selected also allows PEG qualities according to USP to be obtained which are colorless. This is generally the case for color numbers of <10 APHA.

The invention is illustrated by the examples which follow.

### EXAMPLE 1 (COMPARATIVE)

900 g of triethylene glycol (carbonyl content as acetaldehyde 400 ppm) are charged into a pressure vessel with 1.2 g of KOH. This mixture is then reacted with 300 g of ethylene oxide at from 120 to 130° C. On completion of the reaction, the product is discharged under nitrogen and analyzed: Hazen color number 354 APHA.

### EXAMPLE 2 (COMPARATIVE)

300 g of TEG (carbonyl content as acetaldehyde 25 ppm) are charged into a pressure vessel with 1.33 g of 30% sodium methoxide in methanol. Methanol is removed at 80° C. under reduced pressure (20 mbar). The mixture is then reacted with 100.3 g of ethylene oxide at from 120 to 130° C. On completion of the reaction, the product is discharged under nitrogen and analyzed: Hazen color number 22 APHA.

### EXAMPLE 3

300 g of TEG (carbonyl content as acetaldehyde 4000 ppm) are charged into a pressure vessel with 1.50 g of borol solution (aqueous solution of approx. 12% of NaBH4 and approx. 40% of NaOH). Water is removed at 80° C. under reduced pressure (20 mbar). The mixture is then reacted with 100.3 g of ethylene oxide at from 120 to 130° C. On completion of the reaction, the product is discharged under nitrogen and analyzed: Hazen color number 12 APHA.

### EXAMPLE 4

600 g of TEG (carbonyl content as acetaldehyde 75 ppm) are charged into a pressure vessel with 0.72 g of borol solution. Water is removed at 100° C. under reduced pressure (20 mbar). The mixture is then reacted with 100.3 g of ethylene oxide at from 120 to 130° C. On completion of the reaction, the product is discharged under nitrogen and analyzed: Hazen color number 6 APHA. GC analysis: MEG<0.05%, DEG<0.05%, sulfate ash 0.08%, appearance: clear, viscosity: 4.43 mm2/s (98.9° C.), OH number: 557 mg of KOH/g.

### EXAMPLE 5

300 g of TEG (carbonyl content as acetaldehyde 72 ppm) are charged into a pressure vessel with 0.09 g of borol solution. Water is removed at 100° C. under reduced pressure (5 mbar). The mixture is then reacted with 100.3 g of ethylene oxide at from 120 to 130° C. On completion of the reaction, the product is discharged under nitrogen and analyzed: Hazen color number 2 APHA.

### EXAMPLE 6

300 g of TEG (carbonyl content as acetaldehyde 72 ppm) are charged into a pressure vessel with 0.18 g of borol solution. Water is removed at 100° C. under reduced pressure (5 mbar). The mixture is then reacted with 100.3 g of ethylene oxide at from 145 to 155° C. On completion of the reaction, the product is discharged under nitrogen and analyzed: Hazen color number 6 APHA.

All color numbers mentioned relate to a 25% solution of the products in water.

1-14. (canceled)

15. A process for preparing polyoxyalkylene glycol of high purity and having a low color number, comprising reacting the corresponding alkylene oxide and a starter, in the presence of a basic catalyst, and
wherein a reducing agent is present in the polymerization.

16. The process as claimed in claim 15, wherein the starter is of technical grade quality.

17. The process as claimed in claim 15, wherein the reducing agent is added before, or at the beginning of, the polymerization.

18. The process as claimed in claim 17, wherein the reducing agent is added before the polymerization.

19. The process as claimed in claim 15, wherein the reducing agent is selected from complex hydrides, BH3, alkylboranes and hydrogen in combination with hydrogenation catalysts known to those skilled in the art, or mixtures thereof.

20. The process as claimed in claim 19, wherein the reducing agent is selected from borohydrides or aluminohydrides.

21. The process as claimed in claim 20, wherein the reducing agent is selected from LiAlH4, NaBH4, LiBH4 or KBH4.

22. A process as claimed in claim 15, wherein the amount of reducing agent is from 0.002 to 0.06% by weight.

23. The process as claimed in claim 22, wherein the amount of reducing agent is from 0.002 to 0.02% by weight.

24. The process as claimed in claim 22, wherein the amount of reducing agents is from 0.004 to 0.02% by weight.

25. The process as claimed in claim 15, wherein the basic catalyst is selected from hydroxides or alkoxides of the alkali metals and alkaline earth metals.

26. The process as claimed in claim 15, wherein the basic catalyst is added in an amount of from 0.001 to 5% by weight.

27. The process as claimed in claim 26, wherein the basic catalyst is added in an amount of from 0.01 to 1% by weight.

28. The process as claimed in claim 15, wherein the reducing agent includes an amount of base, or has basic properties itself.

29. The process as claimed in claim 28, wherein the reducing agent is selected from KBH4 or NaBH4 in alkali-stabilized aqueous solution.

30. The process as claimed in claim 28, wherein the reducing agent is selected from KBH4 or NaBH4 in alkali-stabilized aqueous solution with NaOH or KOH.

31. The process as claimed in claim 15, wherein the starter is selected from the group consisting of monoethylene glycol MEG, diethylene glycol DEG, triethylene glycol TEG, monopropylene glycol MPG, dipropylene glycol DPG, tripropylene glycol TPG, monobutylene glycol and dibutylene glycol.

32. The process as claimed in claim 15, wherein the polyoxyalkylene glycol is polyethylene glycol.

33. The process as claimed in claim 32, wherein the starter is triethylene glycol.

34. The process as claimed in claim 33, wherein the polyethylene glycol obtained, has a molecular weight of from 150 to 500 g/mol.

35. The process as claimed in claim 33, wherein the polyethylene glycol obtained, has a molecular weight of from 190 to 300 g/mol.

36. The process as claimed in claim 33, wherein the polyethylene glycol obtained, has a molecular weight of from 190 to 250 g/mol.

37. The process as claimed in claim 33, wherein the starter has carbonyl contents of >25 ppm.

38. A method for producing food products, comprising adding the polyethylene glycol obtained by the process of claim 32 to a food product.

39. A method for producing pharmaceutical products, comprising adding the polyethylene glycol obtained by the process of claim 32 to a pharmaceutical product.
